(19) United States
(12) Patent Application Publication
(10) Pub. No.: US 2013/0218048 A1
(43) Pub. Date: Aug. 22, 2013

(54) Apparatus and method for determining a biochemical function of a fluid

(71) Applicants: Oliver Hayden, Herzogenaurach (DE); Manfred Rührig, Lauf a.d. Pegnitz (DE)

(72) Inventors: Oliver Hayden, Herzogenaurach (DE); Manfred Rührig, Lauf a.d. Pegnitz (DE)

(21) Appl. No.: 13/765,809

(22) Filed: Feb. 13, 2013

(30) Foreign Application Priority Data
Feb. 16, 2012 (DE) 102012202336.4

(51) Int. Cl. G01N 33/86; A61B 5/15; A61B 5/157; A61B 5/153

(52) U.S. Cl. CPC G01N 33/86; A61B 5/153; A61B 5/15003; A61B 5/157; A61B 5/150221; A61B 5/150251; A61B 5/150099
USPC 600/577; 600/576; 600/573; 436/63; 422/73

(57) ABSTRACT

Sensor apparatus (1) for determining at least one biochemical function of a fluid (F) with at least one magnetoelastic capillary tube (2) through which the fluid (F) is conveyed, wherein a resonant frequency (fR) of the magnetoelastic capillary tube (2), which depends on a surface loading of the inner wall of the magnetoelastic capillary tube (2) by the fluid (F) conveyed through the tube is able to be read out in a non-contact manner for determining the biochemical function of the fluid (F).

This application claims the benefit of Germany Patent Application No. 102012202336.4 filed on Feb. 16, 2012, the disclosure of which is incorporated, in its entirety, by this reference.

The invention relates to an apparatus and a method for determining a biochemical function of a fluid, especially of a bodily fluid.

In many applications it is necessary to determine biochemical functions of a fluid. The platelets function of blood represents an example of such a function, wherein platelets play a role in diseases of the heart and circulation. These same mechanisms which, on escape of blood from an injured vessel, close up said vessel again, can be responsible for the occurrence of thromboembolic vessel blockages. Different measurement methods are known for determining a platelet function. For example in a platelet function analyzer in accordance with a PFA-100 system with a measurement source, an in-vivo situation of an injured blood vessel can be emulated. In this analyzer a membrane, which can additionally be coated with ADP or Epinephrine, contains a tiny opening through which the introduced blood is sucked with high sheer forces. The platelets adhere in such cases to the membrane and close up the opening. The closure formation is characterized by a pressure measurement. The duration in minutes until the membrane opening is closed off by the platelet clot serves in such cases as a measure for the platelet function. The coagulation system of an organism is highly complex and comprises a plurality of biochemical reactions. A disadvantage of conventional systems, especially also of the conventional platelet function analyzer, lies in the fact that a multiplexing by parallel measurements of different biochemical reactions is not possible or is only possible at considerable expense. In addition it is necessary with the platelet function analyzer to dispense the withdrawn blood in pipettes. This is naturally relatively complex and can also only be carried out by skilled persons.

It is thus the object of the present invention to create an apparatus and a method for determining at least one biochemical function of a fluid, which can determine the biochemical functions of the fluid safely and with little effort.

This object is achieved in accordance with the invention by a sensor apparatus with the features claimed in claim 1.

The invention accordingly creates a sensor apparatus for determining a biochemical function of a fluid with at least one magnetoelastic capillary tube, through which the fluid is conveyed, wherein a resonant frequency of the magnetoelastic capillary tube, which depends on a surface loading of the inner wall of the magnetoelastic capillary tube by the fluid conveyed through it, is able to be read out in a non-contact manner to determine the biochemical function of the fluid.

In a possible embodiment of the inventive sensor apparatus, said apparatus has a transmit coil which excites the magneto-elastic capillary tube into mechanical vibrations in a non-contact manner.

In a further possible embodiment of the inventive sensor apparatus, said apparatus additionally has a pickup coil, which detects a magnetic field generated by the magnetoelastic capillary tube for determining its resonant frequency.

In a possible embodiment of the inventive sensor apparatus the transmit coil and the pickup coil are wound around the magnetoelastic capillary tube.

In a possible embodiment the transmit coil in this case is wound around the magnetoelastic capillary tube and the pickup coil for its part is wound around the transmit coil.

In a further possible embodiment of the inventive sensor apparatus a number of magnetoelastic capillary tubes are provided, which each have a different modulus of elasticity.

In a further possible embodiment of the inventive sensor apparatus the magnetoelastic capillary tubes have a different length.

In a further possible embodiment of the inventive sensor apparatus the magnetoelastic capillary tubes have a different diameter.

In a further possible embodiment of the inventive sensor apparatus the magnetoelastic capillary tubes have a different surface coating which have different supplementary reagents for determining different biochemical functions of the fluid.

In a possible embodiment of the inventive sensor apparatus the magnetoelastic capillary tube consists of a transparent material, for example glass or a transparent plastic.

In a possible embodiment of the inventive sensor apparatus the modulus of elasticity of the magnetoelastic capillary tube is able to be set by pre-magnetization.

In a further possible embodiment of the inventive sensor apparatus the magnetoelastic capillary tube the magnetoelastic capillary tube consists of a material with a high magnetostriction constant.

In a possible embodiment of the inventive sensor apparatus the magnetoelastic capillary tube consists of CoFe alloys.

In an alternate embodiment of the inventive sensor apparatus the magnetoelastic capillary tube consists of highly magnetostrictive rare earth iron alloys.

In a further possible embodiment the magnetoelastic capillary tube of the sensor apparatus consists of magnetostrictive amorphous alloys.

In a further possible embodiment of the inventive sensor apparatus a platelet function of blood is determined as a biochemical function by the sensor apparatus.

The invention further creates a small blood withdrawal tube with a sensor apparatus integrated therein for determining at least one biochemical function of the fluid, wherein the sensor apparatus integrated into the small blood withdrawal tube has at least one magnetoelastic capillary tube through which the fluid is conveyed, wherein a resonant frequency of the magnetoelastic capillary tube which depends on a surface loading of the inner wall of the magnetoelastic capillary tube by the fluid conveyed through it is able to be read out for determining the biochemical function of the fluid in a non-contact manner.

In a possible embodiment of the inventive small blood removal tube a needle is attached to the small blood removal tube, through which blood from a vein is able to be withdrawn from an organism and arrives in a first blood receiving chamber of the small blood removal tube.

In a further possible embodiment of the inventive small blood removal tube with integrated sensor apparatus, the integrated sensor apparatus is separated from the first blood receiving chamber by a first rupture disk or a valve.

In a further possible embodiment of the inventive small blood removal tube the blood conveyed through the magnetoelastic capillary tube of the sensor apparatus arrives in a second blood receiving chamber of the small blood removal tube to which a vacuum is able to be applied.

In a further possible embodiment of the inventive small blood removal tube a second rupture disk or valve is provided between the sensor apparatus and the second blood receiving chamber.

The invention further creates a method with the features specified in claim 16.

The invention accordingly creates a method for determining at least one biochemical function of the fluid, wherein a resonant frequency of a magnetoelastic capillary tube through which the fluid is conveyed is read out for determining the biochemical function of the fluid in a non-contact manner, wherein the resonant frequency of the magnetoelastic capillary tube depends on a surface loading of the inner wall of the magnetoelastic capillary tube by the fluid conveyed through it.

Further possible embodiments of the inventive method and of the inventive apparatus for determining a biochemical function of a fluid are described below, with reference to the enclosed figures, in which:

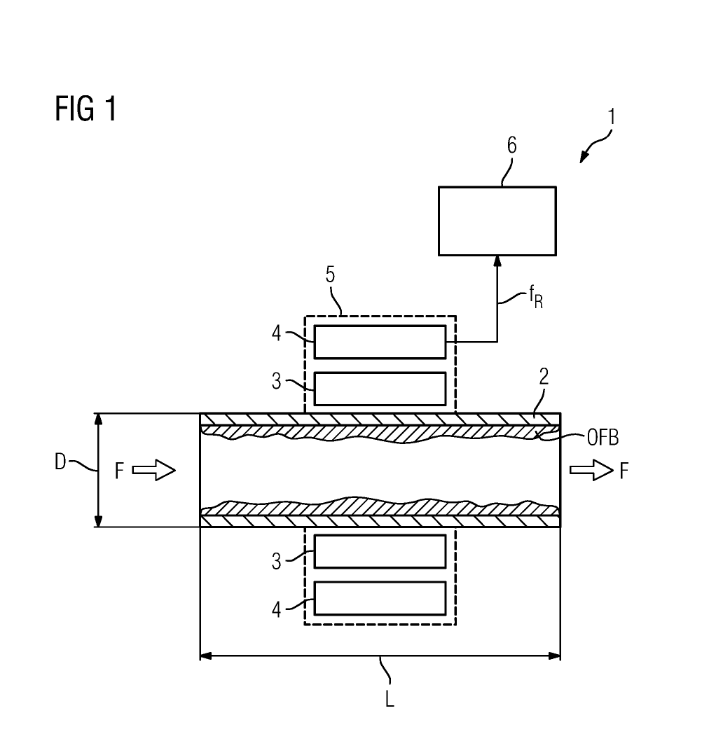

FIG. 1 shows a diagram for presenting the basic method of operation of the inventive method and of the inventive apparatus for determining a biochemical function of a fluid;

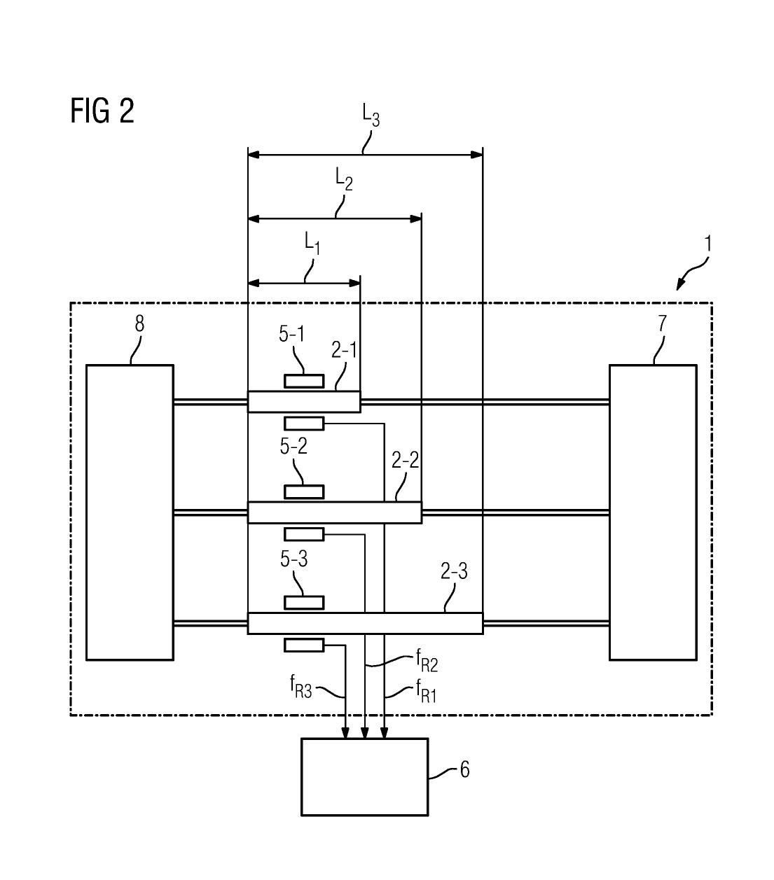

FIG. 2 shows a block diagram for presenting an exemplary embodiment of the inventive sensor apparatus for determining a biochemical function of a fluid.

As can be seen from FIG. 1, in the inventive sensor apparatus 1 a fluid F is conveyed through a magnetoelastic capillary tube 2. The fluid F can involve a bodily fluid, for example urine or blood from a vein. The magnetoelastic capillary tube 2 forms a flexible vibrator of which the mechanical resonant frequency depends on the length L of the magnetoelastic capillary tube 2 as well as a diameter D of the capillary tube 2. Other components of the resonant frequency are the elastic properties in the form of the modulus of elasticity (tensile modulus, coefficient of elasticity or Young's modulus) of the material. The modulus of elasticity or E modulus depends on the environment of the capillary tube, especially on the surface loading OFB of the inner wall of the capillary tube 2 by the substances which adhere to the inner wall of the capillary tube 2 or which accumulate there. Furthermore the E-modulus E of the capillary tube 2 or of the flexible vibrator can depend on the viscosity of the fluid F flowing through the tube. In addition the modulus of elasticity E of the capillary tube depends via the magnetostrictive properties of the material from which the capillary tube 2 is manufactured, on the magnetization state of the capillary tube 2. Thus for example the E modulus of the magnetoelastic capillary tube 2 can be set via a pre-magnetization of the magneto-restrictive material. In a possible embodiment of the inventive sensor apparatus 1 the magnetoelastic capillary tube 2 consists of a material with a high magnetostriction constant. For example the magnetoelastic capillary tube 2 consists of a highly magneto-restrictive Co—Fe alloy. In a further possible embodiment the magnetoelastic capillary tube 2 can feature a highly magnetostrictive rare earth-iron alloy (e.g. Terfenol® (=TbDyFe)). In a further possible embodiment the magnetoelastic capillary tube 2 consists of a highly magnetostrictive Fe—Ga ally (Galfenol®). A class of materials which is especially suitable for embodying the capillary tube 2 is highly magnetostrictive amorphous Co—Fe alloys, which are quenched in the amorphous state by rapid solidification from the melt. The magneto-mechanical coupling factor, i.e. the coupling between mechanical vibrations of a material and the changes in magnetization of the material coupled thereto via the magnetostriction is especially high precisely in amorphous (glassy) materials because of the large E modulus (mechanical hardness) with simultaneously high magnetostriction and easy magnetizability (magnetic softness).

The cross-section of the capillary tube 2 can be round in a possible embodiment. In alternative embodiments capillary tubes with other cross sections, for example rectangular cross-sections, can also be used.

As can be seen in FIG. 1, a magnetization coil 3 is wound around the magnetoelastic capillary tube 2 which applies a magnetic alternating field of frequency f to the magnetoelastic capillary tube 2 in a non-contact manner. Through the alternating field propagated by the magnetostriction of the capillary material the capillary tube 2 is excited into mechanical vibrations. Wound around the magnetization coil 3 is a pickup coil 4, which inductively detects the changes in magnetization generated by the mechanical vibrations in the magnetoelastic capillary tube 2. If the frequency f of the exciter field is tuned over a specific frequency range fₘᵢₙ<f_{R}<fₘₐₓ the resonant frequency f_{R} of the capillary tube 2 can be determined at which the vibration amplitude is at its maximum. In the embodiment depicted in FIG. 1 the pickup coil 4 is wound around the magnetization coil 3. In a further possible embodiment the magnetization coil 3 and the pickup coil 4 can be provided offset along the capillary tube 2. The magnetization coil 3 and the pickup coil 4 together form a signal pickup 5, which transmits the resonant frequency f_{R} of the capillary tube 2 to an evaluation unit 6. If a surface loading OFB forms on the inner walling of the capillary tube 2, as indicated in FIG. 1, the modulus of elasticity E of the capillary tube 2 and thereby its resonant frequency f_{R} changes. The surface loading OFB can be caused by a biochemical reaction of the fluid F conveyed through the tube. The capillary tube 2 or the flexible bar or flexible vibrator respectively is excited via a magnetic field by means of the magnetization coil 3 into vibrations in a non-contact manner. The excited vibrations of the magnetoelastic capillary tube 2 decay at the end of the pulse in a characteristic manner and in doing so generate magnetic fields (stray fields) which for their part can be detected by the pickup coil 4. By a frequency modulation it is possible to read out the resonant frequencies f_{R} of all capillaries or magnetoelastic capillary tubes 2 disposed in the interrogation field.

In a further possible embodiment the flexible vibrator is excited via a magnetic field by means of the magnetization coil 3 into vibrations in a non-contact manner. The magnetization changes in the capillaries are detected by a pickup coil 4 which is equipped in this case so that the fields of the magnetization coil 3 do not penetrate the pickup coil or compensate for each other. A non-penetration can be realized in such cases by the planes of the two coils 3, 4 being approximately at right angles to one another. A compensation can be realized by the pickup coil 4 consisting of two approximately identical part coils which are connected in series and the windings of which are embodied in opposite directions. Expediently the capillary tube 2 then only passes through one of the two part coils while the other part coil only encloses the air flow.

This leads to the changes in magnetic flux which are caused by the magnetization coil 3 canceling each other out in the two part coils of the pickup coil 4 designed in this way while the magnetization changes in the capillary tube 2, which only passes through one of the two part coils, are detected.

In a possible embodiment a platelet function of blood flowing through the tube can be determined as a biochemical function by the sensor apparatus 1. In a possible embodiment the capillary tube can be coated (e.g. with collagen) for activating the platelets. As an alternative a soluble activator (e.g. ADP) can be added to the blood sample. Preferably capillary tubes which consist of a material with high magnetostriction constants and simultaneously have a large modulus of elasticity E are used as magnetoelastic vibrators or magnetoelastic capillary tubes 2, since these have a high magneto-mechanical coupling factor. This produces a high vibration amplitude with relatively large time constant in the decay signal. The reading out of the resonant frequency f_{R} allows the biochemical function of the fluid F flowing through the tube, for example blood, to be determined in a non-contact manner. The speed of flow of the fluid F flowing through the capillary tube 2 depends on a drop AP between the two ends of the magnetoelastic capillary tube 2, wherein this fall in pressure can be adjustable in a possible embodiment of the inventive method. In addition it is possible for the fluid F to be analyzed, for example blood, to have reagents applied to it by an incubation device disposed before the magnetoelastic capillary tube 2 which enter into a biochemical reaction with the fluid F through which for example the surface loading OFB shown in FIG. 1 is generated, which for its part changes the resonant frequency f_{R} of the magnetoelastic capillary tube 2.

In a possible embodiment a number of magnetoelastic capillary tubes 2 of different length L, diameter D and surface coating, for instance activators for blood coagulation, can be used as the sensor unit. In a possible embodiment the capillary tubes 2 have resonant frequencies f_{R} in the Kilohertz range. Furthermore it is possible for a number of capillary tubes or an array of capillary tubes 2 to be integrated into one small blood withdrawal tube.

FIG. 2 shows an exemplary embodiment in which the sensor apparatus 1 has a number of magnetoelastic capillary tubes 2-1, 2-2, 2-3 which are disposed between the first fluid receiving chamber 7 and a second fluid receiving chamber 8. In the exemplary embodiment depicted in FIG. 2 the different capillary tubes have different lengths L1, L2, L3 thus different moduli of elasticity E. The diameters D and the materials of the different capillary tubes 2 can also be different. The sensor apparatus 1 depicted in FIG. 2 can be integrated into a small blood withdrawal tube, wherein the first fluid receiving chamber 7 is supplied with a bodily fluid via a needle. The result achieved by applying a corresponding drop in pressure between the first fluid receiving chamber 7 and the second fluid receiving chamber 8 is that the fluid flows from the first fluid receiving chamber 7 through the capillary tubes 2-1, 2-2, 2-3 disposed in parallel through to the second fluid receiving chamber 8. In a possible embodiment a vacuum can be applied to the second fluid receiving chamber 8 so that a drop in pressure arises between the first and second fluid receiving chambers. The resonant frequencies fRᵢ determined by the various signal pickups 5-i can, as shown in FIG. 2, be supplied for further evaluation to an external evaluation unit 6. In a possible embodiment the evaluation unit 6, for evaluating the measured resonant frequencies fRᵢ, can likewise be integrated into the small blood withdrawal tube. A needle can be attached to the small blood withdrawal tube through which blood from a vein can be removed from an organism and arrives in the first blood receiving chamber 7 of the small blood withdrawal tube. In a possible embodiment the integrated sensor apparatus, i.e. the capillary tubes 2-i disposed in parallel, are separated from the first blood receiving chamber 7 by rupture disks. The second blood receiving chamber 8 forms a waste compartment in which the blood conveyed through the capillary tubes 2-i is received without it getting out. Rupture disks can also be provided between the two blood receiving chambers 8 and the integrated sensor apparatus, which rupture on application of a corresponding vacuum, so that the fluid F flows through the capillary tubes 2-i. As can be seen from FIG. 2, the parallel arrangement of the different magnetoelastic capillary tubes 2-i, which each have different moduli of elasticity but also different incubation devices or coatings, enables a number of biochemical functions of the fluid F to be detected simultaneously. A sensor array or a sensor field with a number of capillary tubes 2-i disposed in parallel can be integrated in a simple manner into a device in a space-saving way, especially into a small blood withdrawal tube. The detected resonant frequencies f_{Ri} can be transferred via a data interface wirelessly or by wire to the external evaluation unit 6. The inventive sensor apparatus 1 can also be integrated into an exchangeable cassette or the like. By changing or varying the diameter D of the different capillary tubes 2, the fluid F can also have different shear forces applied to it, which can for example play a role in the measurement of the blood coagulation. As a result of the plurality of parallel-integrated capillary tubes 2-i the inventive sensor apparatus 1 to some extent provides a multiplex function for simultaneous or parallel measurement of a plurality of different biochemical functions of the fluid F to be investigated. In this case the different capillary tubes 2 are able to be integrated into the device easily and with a low space requirement. The coating of the different capillary tubes 2 with different reagents allows the widest variety of biochemical functions of the fluid F flowing through the tubes to be detected. Furthermore the variation of the diameter D allows different shear forces to be applied to the fluid F to be examined. The transmit coil and the pickup coil can be wound around the capillary tubes 2-i in a simple manner to save space, through which the integration into a device, for example a small blood withdrawal tube, is additionally facilitated. The investigated fluid F can be stored after the investigation has been completed for further subsequent measurements in a simple manner in a small blood withdrawal tube into which the inventive sensor apparatus 1 is integrated.

1. A sensor apparatus (1) for determining at least one biochemical function of a fluid (F) with at least one magnetoelastic capillary tube (2), through which the fluid (F) is conveyed, wherein a resonant frequency (fR) of the magnetoelastic capillary tube (2), which depends on a surface loading of the inner wall of the magnetoelastic capillary tube (2) by the fluid (F) conveyed through the tube, is able to be read out in a non-contact manner for determining the biochemical function of the fluid (F).

2. The sensor apparatus as claimed in claim 1, wherein a magnetization coil (3), which excites the magnetoelastic capillary tube (2) into mechanical vibrations in a non-contact manner, and a pickup coil (4) is provided, which inductively detects the changes in magnetization generated in the magnetoelectric capillary tube (2).

3. The sensor apparatus as claimed in claim 1, wherein the magnetization coil (3) and the pickup coil (4) are wound concentrically and the capillary tube (2) penetrates the surfaces delimited by the two coils (3, 4).

4. The sensor apparatus as claimed in claim 1, wherein the coil surfaces of the magnetization coil (3) and the pickup coil (4) enclose an angle.

5. The sensor apparatus as claimed in claim 4, wherein the pickup coil (4) is embodied or oriented such that the magnetic field penetrating it is minimal.

6. The sensor apparatus as claimed in claim 5, wherein a number of magnetoelastic capillary tubes (2) are provided, which each have a different modulus of elasticity.

7. The sensor apparatus as claimed in claim 6, wherein the magnetoelastic capillary tubes (2) have a different length (L) and/or a different diameter (D).

8. The sensor apparatus as claimed in claim 7, wherein the magnetoelastic capillary tubes (2) have a different surface coating, featuring different supplementary reagents for determining different biochemical functions of the fluid (F).

9. The sensor apparatus as claimed in claim 6, wherein the modulus of elasticity of the magnetoelastic capillary tube (2) is able to be set by pre-magnetization.

10. The sensor apparatus as claimed in claim 9, wherein the magnetoelastic capillary tubes (2) is made of a material with a highly magnetostrictive constant, especially highly magnetostrictive CoFe alloys, highly magnetostrictive amorphous Co—Fe alloys, highly magneto-restrictive rare earth iron alloys or highly magneto-strictive Ga—Fe alloys.

11. The sensor apparatus as claimed in claim 10, wherein a platelet function of blood is determined as a biochemical function by the sensor apparatus (1).

12. A small blood withdrawal tube with a sensor apparatus (1) integrated therein, as claimed in claim 11.

13. The small blood withdrawal tube as claimed in claim 12, wherein a needle is attached to the small blood withdrawal tube through which blood from a vein is able to be withdrawn from an organism and arrives in a first blood receiving chamber (7) of the small blood withdrawal tube.

14. The small blood withdrawal tube as claimed in claim 13, wherein the integrated sensor apparatus (1) is separated from the first blood receiving chamber (7) by a first rupture disk or by a valve.

15. The small blood withdrawal tube as claimed in claim 14, wherein the blood conveyed through the magnetoelastic capillary tube (2) of the sensor apparatus (1) reaches a second blood receiving chamber (8) of the small blood withdrawal tube to which a vacuum is able to be applied.

16. The small blood withdrawal tube as claimed in claim 15, wherein a second rupture disk or a valve is provided between the sensor apparatus (1) and the second blood receiving chamber (8).

17. A method for determining at least one biochemical function of a fluid, wherein a resonant frequency (fR) of a magnetoelastic capillary tube (2), through which the fluid (F) is conveyed, is read out in a non-contact manner to determine the biochemical function of the fluid (F), wherein the resonant frequency (fR) of the magnetoelastic capillary tube (2) depends on a surface loading of the inner wall of the magnetoelastic capillary tube (2) by the fluid (F) passing through it.
